(19) United States
(12) Patent Application Publication
(10) Pub. No.: US 2012/0216678 A1
(43) Pub. Date: Aug. 30, 2012

(54) ABSORBENT COMPOSITION AND PROCESS FOR REMOVING CO2 AND/OR H2S FROM A GAS COMPRISING CO2 AND/OR H2S

(76) Inventors: Frank Haiko Geuzebroek, Amsterdam (NL); Armin Schneider, Amsterdam (NL); Renze Wijntje, Amsterdam (NL); Xiaohui Zhang, Amsterdam (NL)

(21) Appl. No.: 13/389,812

(22) PCT Filed: Aug. 11, 2010
(86) PCT No.: PCT/EP2010/061697

§ 371 Date: May 16, 2012

(30) Foreign Application Priority Data
Aug. 11, 2009 (EP) 09167642.9

(51) Int. Cl. B01D 53/14; C07C 215/06; C09K 3/00

(52) U.S. Cl. 95/187; 252/190; 564/503; 95/235; 95/236

(57) ABSTRACT

An absorbent composition for removing CO₂ and/or H₂S from a gas comprising a polyamine, a monoamine and water, wherein the polyamine comprises a polyamine having 3 to 5 amine functions and has a molecular weight of less than 200 g/mol; wherein the monoamine comprises a tertiary monoamine; and wherein the weight ratio of the polyamine having 3 to 5 amine functions to the tertiary monoamine is more than 1:1. A process wherein such an absorbent composition is used and a use of a tertiary monoamine as an accelerator for accelerating the removal of CO₂ and/or H₂S from a CO₂ and/or H₂S containing polyamine having 3 to 5 amine functions.

### FIELD OF THE INVENTION

The invention relates to an absorbent composition and a process for removing carbon dioxide (CO₂) and/or hydrogen sulfide (H₂S) from a gas comprising CO₂ and/or H₂S.

### BACKGROUND OF THE INVENTION

During the last decades there has been a substantial global increase in the amount of CO₂ emission to the atmosphere. Emissions of CO₂ into the atmosphere are thought to be harmful due to its “greenhouse gas” property, contributing to global warming. Following the Kyoto agreement, CO₂ emission has to be reduced in order to prevent or counteract unwanted changes in climate. Sources of CO₂ emission include the combustion of fossil fuels, for example coal or natural gas, for electricity generation and the use of petroleum products as a transportation and heating fuel. These processes result in the production of gases comprising CO₂. Thus, removal of at least part of the CO₂ from these gases prior to emission of these gases into the atmosphere is desirable.

In addition, there is a desire to limit and reduce H₂S emission into the environment.

The removal of CO₂ and/or H₂S from a gas comprising CO₂ and/or H₂S can be carried out by using an absorbent composition to absorb the CO₂ and/or H₂S from the gas to generate a CO₂ and/or H₂S lean gas and a CO₂ and/or H₂S rich absorbent composition. The CO₂ and/or H₂S rich absorbent composition can be regenerated, for example by stripping, to generate a CO₂ and/or H₂S rich gas and a CO₂ and/or H₂S lean absorbent composition, whereafter the CO₂ and/or H₂S lean absorbent composition can be recycled.

Processes for the removal of CO₂ and/or H₂S are known in the art. For example, in US2006/0104877, a method of deacidizing a gaseous effluent comprising an acid compound such as carbon dioxide or hydrogen sulfide is described, wherein the gaseous effluent is contacted with an absorbent solution so as to obtain a gaseous effluent depleted in acid compounds. The absorbent solution can consist of one or more compounds reactive with or having a physico-chemical affinity with the acid compound and possibly one or more solvation compounds. As reactive compounds alkanolamines and polyamines are mentioned. A list of more than 80 possible reactive compounds is described, mentioning in passing N,N-dimethylethanolamine, N,N-dimethyldipropylenetriamine and diethylenetriamine. The reactive compounds are said to represent 10 to 100% by weight of the absorbent, preferably 25 to 90% by weight and ideally 40 to 80% by weight. The absorbent solution is stated to possibly also contain one or more activators for favouring absorption of the compounds to be treated. As examples amines are mentioned. A list of about 80 possible activators is described, mentioning in passing N,N-dimethyldipropylenetriamine. The list of possible activators does not include any tertiary amines. The activator concentration is stated to range between 0 and 30% by weight, preferably between 0 and 15% by weight of the absorbent solution.

EP2036602 describes an absorbent liquid and a method for removing CO₂ or H₂S from a gas with use of such absorbent liquid wherein the absorbent liquid comprises a first compound component and a second compound component. The first compound component is represented by a series of three formulae of nitrogen-containing compounds, of which each formula I and II comprises one tertiary amine function. The second compound component is said to include a nitrogen-containing compound having in a molecule thereof at least two members selected from a primary nitrogen, a secondary nitrogen and a tertiary nitrogen, or a nitrogen containing compound having in a molecule thereof all of primary, secondary and tertiary nitrogens. As an example of the first compound component 2-dimethylaminoethanol is mentioned. For the second compound component some 10 different possible chemical formulae are mentioned, each formulae covering a wide range of possible nitrogen containing compounds. Ring-shaped compounds are indicated to be preferred. The first compound component is said to preferably be contained in an amount in a range from equal to or larger than 15 wt % to equal to or less than 45 wt % and the second compound component is said to be preferably contained in a similar amount. The total amount of the first compound component and the second compound component are stated to preferably be more than 30 wt % to equal or less than 90 wt %.

In a presentation of Peter Bruder given at the joint seminar on CO₂ absorption fundamentals NTNU in Trontheim on 15 Jun. 2009 (published via the internet website of the Norges teknisk-naturvitenskapelige universitet), a system comprising N,N-dimethylethanolamine (DMMEA) and methylaminopropyleneamine (MAPA) is described for the absorption of CO₂. Although, in passing, a system is described comprising 5 M methylaminopropyleneamine and 3 M N,N-dimethylethanolamine, it is concluded that, if N,N-dimethylethanolamine and methylaminopropyleneamine are present, systems with high N,N-dimethylethanolamine and low concentration of methylaminopropyleneamine have the highest cyclic capacity per kg solution. If anything, the presentation therefore appears to teach towards an excess of N,N-dimethylethanolamine over methylaminopropylene-amine.

The regeneration of the CO₂ and/or H₂S rich absorbent solution can require an extensive amount of energy.

It would be desirable to provide an absorbent composition and a process that allows for regeneration of the CO₂ and/or H₂S rich absorbent solution that uses a minimal amount of energy, whilst on the other hand obtaining a good removal of carbon dioxide from CO₂ and/or H₂S rich absorbent solution.

### SUMMARY OF THE INVENTION

Accordingly the present invention provides an absorbent composition, for removing CO₂ and/or H₂S from a gas comprising CO₂ and/or H₂S, the absorbent composition comprising a polyamine, a monoamine and water,
wherein the polyamine comprises a polyamine having 3 to 5 amine functions and has a molecular weight of less than 200 g/mol;
wherein the monoamine comprises a tertiary monoamine; and wherein the weight ratio of the polyamine having 3 to 5 amine functions to the tertiary monoamine is more than 1:1.

In addition, the present invention provides a process, for removing CO₂ and/or H₂S from a gas comprising CO₂ and/or H₂S, the process comprising the steps of:
(a) contacting the gas in an absorber with an absorbent composition wherein the absorbent composition absorbs at least part of the CO₂ and/or H₂S in the gas, to produce a CO₂ and/or H₂S lean gas and a CO₂ and/or H₂S rich absorbent composition;
(b) removing at least part of the CO₂ and/or H₂S from the CO₂ and/or H₂S rich absorbent composition in a regenerator to produce a CO₂ and/or H₂S rich gas and a CO₂ and/or H₂S lean absorbent composition;
wherein the absorbent composition is an absorbent composition as indicated above.

The invention further provides a use of a tertiary monoamine as an accelerator for accelerating the removal of CO₂ and/or H₂S from a CO₂ and/or H₂S containing polyamine having 3 to 5 amine functions.

The absorbent composition and the process according to the invention advantageously allows for the removal of CO₂ and/or H₂S from a CO₂ and/or H₂S rich absorbent composition by using a minimal amount of energy, whilst on the other hand a good removal of carbon dioxide from CO₂ and/or H₂S rich absorbent solution is still obtained.

### BRIEF DESCRIPTION OF THE DRAWINGS

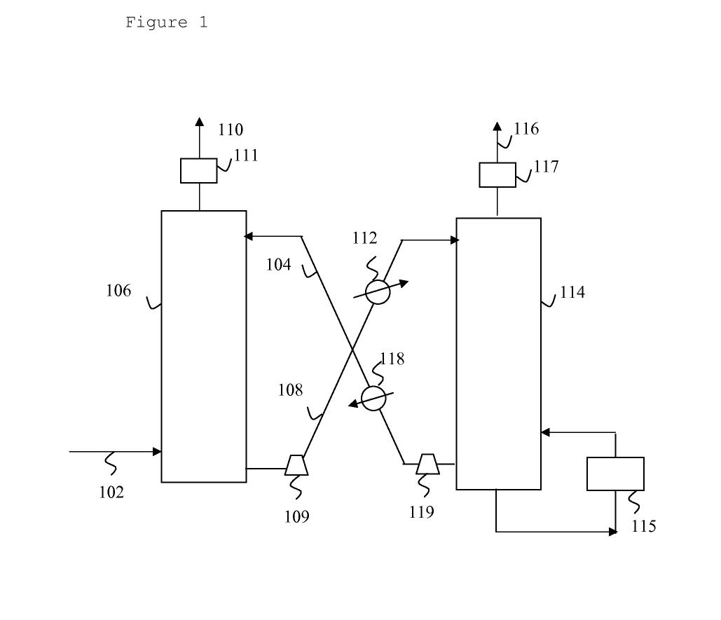

The invention is illustrated with the enclosed FIG. 1 representing a schematic flowchart showing an embodiment of the process of the invention.

### DETAILED DESCRIPTION OF THE INVENTION

As indicated above, the absorbent composition comprises a polyamine, a monoamine and water. The absorbent composition can comprise one or more polyamines, preferably 1 to 4, more preferably 1 to 3 and most preferably 1 or 2 polyamines. At least one of the polyamines comprises a polyamine having 3 to 5 amine functions. By an amine function is understood a group comprising a nitrogen atom. Amine functions are sometimes also referred to as amino groups, amine groups or nitrogen-containing groups. The absorbent composition can comprise one or more polyamines having 3 to 5 amine functions and preferably comprises 1 to 4, more preferably 1 to 3 and most preferably 1 or 2 polyamines having 3 to 5 amine functions.

The polyamines have a molecular weight of less than 200 g/mol, preferably of less than 190 g/mol. Preferably, the polyamines have a weight of more than 50 g/mol. The advantage over polyamines having a higher weight is that the absorbent has a lower viscosity and is easier to handle.

Preferably the polyamine having 3 to 5 amine functions is a non-cyclic polyamine having 3 to 5 amine functions.

More preferably the polyamine having 3 to 5 amine functions is a polyamine of formula I:

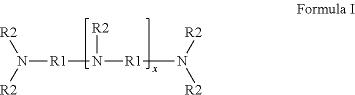

wherein each R1 independently represents a substituted or non-substituted alkylene group comprising 1 to 6 carbon atoms; wherein each R2 independently represents hydrogen or a hydrocarbon group comprising 1 to 12 carbon atoms; and wherein x can be 1, 2 or 3.

It is to be understood that each R1 independently can represent a different substituted or non-substituted alkylene group comprising 1 to 6 carbon atoms. For example, one R1 group can be a non-substituted alkylene group of 2 carbon atoms and the other R1 group can be an oxygen substituted alkylene group having 3 carbon atoms. Preferably each R1 independently represents a substituted or non-substituted alkylene group comprising 2 to 6, more preferably 2 to 4 carbon atoms. If substituted, a R1 group is preferably substituted with an oxygen containing group such as a ketone or hydroxyl group. Preferably the R1 alkylene groups are non-substituted. More preferably each R1 independently represents a methylene, ethylene, propylene, tetramethylene or pentamethylene group, more preferably an ethylene or propylene group and most preferably a propylene group.

It is to be understood that each R2 independently can represent a different group. For example, one R2 group can be a hydrogen group and another R2 group can be a hydrocarbon group such as an ethyl, ethoxy or hydroxyethyl group. By a hydrocarbon group is understood a group comprising both hydrogen as well as carbon atoms. Examples of hydrocarbon groups include alkyl groups, alkyloxy groups, hydroxyalkyl groups and carboxyl groups. Preferably each R2 independently represents hydrogen or a substituted or non-substituted hydrocarbon group comprising 2 to 6 carbon atoms, more preferably 2 to 4 hydrocarbon atoms. More preferably each R2 independently represents hydrogen, a hydroxyl group or a methyl, ethyl, n-propyl, iso-propyl, n-butyl, iso-butyl, tert-butyl, pentyl, hexyl, heptyl, octyl, nonyl, decyl, undecyl, dodecyl, methyloxy, ethyloxy, n-propyloxy, iso-propyloxy, hydroxymethyl, hydroxyethyl, or hydroxypropyl group, more preferably hydrogen or a methyl or ethyl group and most preferably hydrogen or a methyl group.

In a further preferred embodiment at most 1 or 2, more preferably at most 1 of the R2 groups represents a hydroxyl group, such that the polyamine having 3 to 5 amine groups comprises at most one or two, more preferably at most one hydroxyl group.

Preferably x is 1 or 2, that is, preferably the polyamine comprises a polyamine having 3 or 4 amine functions. Most preferably x is 1 and the polyamine comprises a polyamine having 3 amine functions.

In a preferred embodiment the polyamine having 3 to 5 amine functions comprises at least one secondary amine function. More preferably the polyamine comprises at least one tertiary amine function and least one secondary amine function. Most preferably the polyamine comprises at least one tertiary amine function at least one secondary amine function and at least one primary amine function.

Examples of polyamines having 3 to 5 amine functions that can be used in the absorbent composition or process according to the invention include: N-(2-aminoethyl)-1,3-propanediamine, Dipropylenetriamine (N-(3-aminopropyl)1,3-propanediamine), Spermidine (N-(4-aminobutyl)-1,3-propanediamine), N,N-Dimethylaminopropylaminopropylamine, Diethylenetriamine (N-(2-aminoethyl)-1,2-ethanediamine), N,N-dimethyldiethylenetriamine, N,N,N′,N″,N″-pentamethyldiethylenetriamine. N,N,N′,N″,N″-pentamethyldipropylenetriamine (N-(3-(dimethylamino)propyl)-N,N′,N′-trimethylpropane-1,3-diamine), N,N,N″,N″-tetramethyldipropylenetriamine, Spermine (N-(3-aminopropyl)dipropylenetriamine), tris(2-aminoethyl)amine, Triethylenetetramine, N,N-dimethyltriethylenetetramine, tetraethylenepentamine and mixtures thereof. Preferably the polyamine having 3 to 5 amine functions comprises diethylenetriamine, dimethylaminopropylaminopropylamine or a combination thereof.

In addition to the polyamine having 3 to 5 amine functions other polyamines may be present in the absorbent composition. Other polyamines that may additionally be used in the absorbent composition or process according to the invention include methylaminopropyleneamine, piperazine, N,N′-Dimethylpiperazine, N,N′-Diethylpiperazine, N,N′-Diethanolpiperazine, N,N,N′,N′-Tetraethyl-ethylenediamine, N,N,N′,N′-Tetramethyl-1,3-propanediamine, N,N,N′,N′-Tetraethyl-propanediamine, N,N,N′,N′-Tetramethyl-1,4-butanediamine and mixtures thereof.

The absorbent composition can comprise one or more monoamines, preferably 1 to 4, more preferably 1 to 3 and most preferably 1 or 2 monoamines.

At least one of the monoamines comprises a tertiary monoamine. The absorbent composition can comprise one or more tertiary monoamines and preferably comprises 1 to 4, more preferably 1 to 3 and most preferably 1 or 2 tertiary monoamines.

Preferably the tertiary monoamine is a non-cyclic tertiary monoamine.

More preferably the tertiary monoamine is a monoamine of formula II:

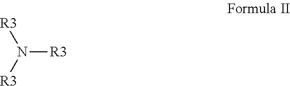

wherein each R3 independently can represent a hydrocarbon group comprising 1 to 6 carbon atoms.

It is to be understood that each R3 independently can represent a different group. For example, one R3 group can be a methyl group and another R3 group can be an ethyl, ethoxy or hydroxyethyl group. Examples of hydrocarbon groups that can be used as R3 include alkyl groups, alkyloxy groups, hydroxyalkyl groups and carboxyl groups. Preferably each R3 independently represents a hydroxyl or a substituted or non-substituted hydrocarbon group comprising 2 to 6 carbon atoms, more preferably 2 to 4 hydrocarbon atoms.

Preferably each R3 independently represents a hydroxyl, methyl, ethyl, propyl, iso-propyl n-butyl, iso-butyl, tert-butyl, pentyl, methyloxy, ethyloxy, propyloxy, iso-propyloxy, methylsulfanyl, ethylsulfanyl, propylsulfanyl or isopropylsulfanyl group. Most preferably each R3 independently represent a methyl, ethyl, methyloxy or ethyloxy group.

In a preferred embodiment at most 1 or 2, more preferably at most 1 of the R3 groups represents a hydroxyl group, such that the tertiary monoamine comprises at most one or two, more preferably at most one hydroxyl group.

In another preferred embodiment, one R3 comprises an alkyloxy or hydroxyalkyl group comprising 1 to 3 carbon atoms and the other R3 groups independently comprise an alkylgroup comprising 1 or 2 carbon atoms.

Examples of tertiary mono-amines that can be used in the absorbent composition or process according to the invention include Dimethylaminoethanol, N,N-Diethylethanolamine, 1-Diethylamino-2-propanol, 1-Dimethylamino-2-propanol, 3-Dimethylamino-1-propanol, 3-Diethylamino-1-propanol, 3-Diethylamino-1,2-propanediol, 2-Ethylmethylamino-1-ethanol, 2-Dipropylamino-1-ethanol, Methyldiethanolamine, Dimethylpropylamine, N-methyldibutylamine, Dimethylcyclohexylamine, N,N,-diethylhydroxylamine, Diisopropylethylamine, 4-(diethylamino)-2-butanol, 4-(dipropylamino)-2-butanol, 4-(propylisopropylamino)-2-butanol or mixtures thereof.

Preferably the tertiary monoamine comprises N,N-dimethylmonoethanolamine, N,N-diethylmonoethanolamine, or a combination thereof. Most preferably the tertiary monoamine is dimethylmonoethanolamine.

In addition to the tertiary monoamine other monoamines may be present in the absorbent composition. Other monoamines that may additionally be used in the absorbent composition or process according to the invention include Aminomethylpropanol, 2-Amino-2-methyl-1,3-propandiol, Methylcyclohexylamine, Diethanolamine, 1-amino-2-propanol 2-amino-2-methyl-1,3-propanediol, 4-(propylamino)-2-butanol, 4-(isopropylamino)-2-butanol or mixtures thereof.

The weight ratio of the polyamine component having 3 to 5 amine functions to the tertiary monoamine component is more than 1:1. Preferably the weight ratio of the polyamine component having 3 to 5 amine functions to the tertiary monoamine component lies in the range from more than 1:1 to 5:1, more preferably in the range from more than 1:1 to 3:1, and most preferably in the range from more than 1:1 to 2:1. The advantage of an equal to or higher amount of polyamine over tertiary monoamine is that a higher cyclic capacity per kg solution might be obtained.

The polyamine component having 3 to 5 amine functions is preferably present in the absorbent composition in a concentration in the range from 20 to 65 wt % and more preferably in the range from 25 to 60 wt %.

The tertiary monoamine component is preferably present in the absorbent composition in a concentration in the range from 5-50 wt %, and more preferably in the range from 10 to 45 wt %.

As indicated above, the aqueous absorbent composition comprises a polyamine, a monoamine and water. In a preferred embodiment the total weight percentage of polyamine and monoamine is less than or equal to 70 wt % based on the total absorbent composition, preferably less than or equal to 65 wt %, still more preferably less than or equal to 55 wt % based on the total absorbent composition.

Without wishing to be bound by any kind of theory, it is believed that the polyamine component having 3 to 5 amine functions allows for a higher loading of CO₂ and/or H₂S in the absorbent composition, whereas the tertiary monoamine component allows for an accelerated regeneration of the absorbent composition comprising the polyamine component having 3 to 5 amine functions. The present invention therefore also provides a use of a tertiary monoamine as an accelerator for accelerating a regeneration of a CO₂ and/or H₂S containing polyamine, especially a CO₂ and/or H₂S containing polyamine having 3 to 5 amine functions, to produce a polyamine, especially a polyamine having 3 to 5 amine functions, containing less CO₂ and/or H₂S. In such a regeneration, the bicarbonate (HCO₃⁻¹), which is a typical product in the absorption reaction of CO2 and tertiary monoamine, is first regenerated and tertiary monoamine is turned back to free amine. The concentration of bicarbonate is decreased. To maintain the chemical equilibrium in the system and to compensate for the decrease of bicarbonate ions in the solvent, more bicarbonate ions are formed through the hydrolysis of carbamates, which are ions formed in the absorption reaction of CO2 and polyamine with primary or secondary amino groups. The carbamate hydrolysis is thus accelerated. The regeneration of the polyamine is enhanced by turning carbamate into bicarbonate and further into CO2, instead of directly turning back to CO2 and free polyamine.

Such a tertiary monoamine can also be referred to as a regeneration accelerator as the removal of CO₂ and/or H₂S from a CO₂ and/or H₂S containing polyamine having 3 to 5 amine functions is preferably carried out in a so-called regenerator. Preferably the removal is carried out in the presence of water and the tertiary monoamine and the polyamine having 3 to 5 amine functions is preferably as further described in this patent application. As illustrated above, the tertiary monoamine is preferably present in a weight ratio of the polyamine having 3 to 5 amine functions to the tertiary monoamine of more than 1:1.

The absorbent composition may further contain one or more additional physical solvent compounds. Suitable physical solvent compounds include glycols, polyethylene glycols, polypropylene glycols, ethylene, glycol-propylene glycol copolymers, glycol ethers, alcohols, ureas, lactames, N-alkylated pyrrolidones, N-alkylated piperidones, cyclotetramethylene sulfones, N-alkylformamides, N-alkylacetamides, ether-ketones or alkyl phosphates and derivatives or combinations thereof. Preferred physical solvent compounds include N-methyl-pyrrolidon, tetramethylenesulfon (sulfolane), methanol, dimethylether compounds of polyethylene glycol or combinations thereof. If such an additional physical solvent compound is present, the absorbent composition, preferably comprises in the range from 10 to 70 wt %, preferably 30 to 60 wt % of the additional physical solvent compound.

Furthermore a corrosion inhibitor can added to the absorbent composition. Suitable corrosion inhibitors are described for example in U.S. Pat. No. 6,036,888, US2006/0104877 and US2004/0253159. The use of such a corrosion inhibitor may be especially advantageous when the gas comprising CO₂ and/or H₂S comprises an appreciable quantity of oxygen, suitably in the range of from 1 to 22% (v/v) of oxygen. Furthermore degradation inhibitors and/or foaming inhibitors can be added to the absorbent composition.

The invention further provides a process for the removal of CO₂ and/or H₂S from a gas comprising CO₂ and/or H₂S using the above absorbent composition. Such a process can comprise the steps of
(a) contacting the gas in an absorber with the absorbent composition wherein the absorbent composition absorbs at least part of the CO₂ and/or H₂S in the gas, to produce a CO₂ and/or H₂S lean gas and a CO₂ and/or H₂S rich absorbent composition;
(b) removing at least part of the CO₂ and/or H₂S from the CO₂ and/or H₂S rich absorbent composition in a regenerator to produce a CO₂ and/or H₂S rich gas and a CO₂ and/or H₂S lean absorbent composition.

In a preferred embodiment, the process further comprises an optional step (c) wherein the CO₂ and/or H₂S lean absorbent composition produced in step b) is cooled, and/or a step e) wherein the, optionally cooled, CO₂ and/or H₂S lean absorbent composition is recycled to step a) to be contacted with the gas in the absorber.

The gas comprising CO₂ and/or H₂S that may be used as a feed gas in the process of the invention can be any gas known by the skilled person in the art to comprise CO₂ and/or H₂S. For example the gas comprising CO₂ and/or H₂S may comprise a natural gas, synthetic natural gas, synthesis gas, combustion fumes, refinery gas, Claus tail gas or biomass fermentation gas. The gas preferably comprises in the range from 50 ppmv to 70 vol. %, more preferably from 100 ppmv to 30 vol. % and most preferably from 100 ppmv to 15 vol. % of CO₂ and/or in the range from 10 ppmv to 50 vol. %, more preferably in the range from 50 ppmv to 30 vol. % and most preferably in the range from 50 ppmv to 15 vol. % of H₂S.

In addition to CO₂ and/or H₂S the gas can comprise additional acid compounds, for example SO₂ (sulfurdioxide), mercaptans, COS (carbonylsulfide) or CS₂ (carbondisulfide). These additional acid compounds can also be at least partly removed by the process according to the invention.

The absorber may be any type of absorber known by the skilled person in the art to be suitable to carry out the absorption. For example, the absorber may be an absorber comprising a membrane, which is keeping the gas and absorbent composition separate but allows the absorption of CO₂ and/or H₂S through the membrane.

Preferably, the absorber is operated at a temperature in the range of from 10 to 100° C., more preferably from 20 to 80° C., and still more preferably from 20 to 60° C.

In the process of the invention, the absorber can advantageously operate at a high temperature, such as for example a temperature in the range from 50 to 70° C., whilst still allowing for sufficient removal of CO₂ and/or H₂S. Therefore the process of the invention is especially advantageous in a hot and/or dry climate, for example in a desert, where cooling of the absorber may be expensive.

Preferably the pressure in the absorber is in the range from 1.0 to 110 bar. When the gas comprises synthesis gas, a pressure in the range from 20 to 6o bar may be more preferred. When the gas comprises natural gas, a pressure in the range from 50 to 90 bar may be more preferred.

The regenerator may be any type of regenerator known by the skilled person in the art to be suitable to carry out the regeneration of the CO₂ and/or H₂S rich absorbent composition. For example, the regenerator may be an regenerator comprising a membrane which is keeping for example steam and the CO₂ and/or H₂S rich absorbent composition separate but allows the desorption of CO₂ and/or H₂S through the membrane.

Preferably, the regenerator is operated at a temperature sufficiently high to ensure that a substantial amount of CO₂ and/or H₂S is liberated from the CO₂ and/or H₂S rich absorbent composition. Preferably the regenerator is operated at a temperature in the range from 60 to 170° C., more preferably from 70 to 160° C. and still more preferably from 80 to 140° C.

Preferably the regenerator is operated at a total pressure in the range of from 0.001 bar to 50 bar, more preferably from more than 1.0 to 30 bar, still more preferably from 1.5 to 20 bar, still more preferably from 2 to 10 bar.

The CO₂ and/or H₂S rich gas obtained in step b) can be pressurized in a compressor. If compressed, the CO₂ and/or H₂S rich gas obtained in step b) is preferably compressed to a pressure in the range of from 20 to 300 bar, more preferably in the range of from 40 to 300 bar and most preferably in the range of from 60 to 300 bar. The pressurised CO₂ and/or H₂S rich gas can be used for many purposes, in particular for enhanced recovery of oil, coal bed methane or for sequestration in a subterranean formation. By injecting CO₂ and/or H₂S into an oil reservoir, the oil recovery rate can be increased. For example, the pressurised CO₂ and/or H₂S rich gas is injected into the oil reservoir, where it will be mixed with some of the oil which is present. The mixture of CO₂ and/or H₂S and oil will displace oil, which cannot be displaced by traditional injections.

The invention will now be illustrated, by means of example only, with reference to the accompanying FIG. 1.

In FIG. 1 a stream of feed gas (102) comprising CO₂ and/or H₂S is contacted with a stream of an aqueous absorbent composition (104) comprising a polyamine having 3 to 5 amine functions, a tertiary monoamine and water in an absorber (106) at a temperature of about 40° C. In the absorber, CO₂ and/or H₂S is reacted with the polyamine having 3 to 5 amine functions and the tertiary monoamine in the absorbent composition to produce a stream of CO₂ and/or H₂S rich absorbent composition (108) and a stream of treated CO₂ and/or H₂S lean product gas (110). The stream of treated CO₂ and/or H₂S lean product gas (110) is cooled and/or compressed in a recovery unit (111) to recover water and/or amine from the treated CO₂ and/or H₂S lean product gas (110). The stream of CO₂ and/or H₂S rich absorbent composition (108) is forwarded via pump (109), heated in heat exchanger (112) and subsequently regenerated in regenerator (114) to produce a stream of CO₂ and/or H₂S rich product gas (116) and a stream of regenerated CO₂ and/or H₂S lean absorbent composition (104). The regenerator is kept at a temperature of about 120° C. by reboiler (115). The stream of CO₂ and/or H₂S rich product gas (116) is cooled and/or compressed in a recovery unit (117) to recover water and/or amine from the CO₂ and/or H₂S rich product gas (116). The regenerated CO₂ and/or H₂S lean absorbent composition (104) is cooled in heat exchanger (118) and recycled via pump (119) to absorber (106).

### EXAMPLES 1-3 AND COMPARATIVE EXAMPLES A-F

A feed gas comprising nitrogen loaded with CO₂ was treated with an absorbent composition in a set-up comprising an absorber and a regenerator. The absorbent composition comprised aqueous solutions of the amines as indicated in Table 1. The composition of the feed gas and the feed gas flow are listed in Table 1. In Table I DMAPAPA refers to N,N-dimethyldipropylenetriamine and DMMEA refers to N,N-dimethylmonoethanolamine.

The feed gas was fed via a catch pot into the bottom of the absorber where it was contacted in a countercurrent fashion with the absorbent composition flowing from the top of the absorber to the bottom of the absorber. A CO₂ lean gas was obtained from the top of the absorber. A CO₂ rich absorbent composition was obtained from the bottom of the absorber and forwarded via a pump and an electrical heater to the top of the regenerator. Additional heat was applied to the regenerator by heating the bottom of the regenerator vessel with an electrical heating coil. The energy applied via both electrical heaters was monitored. From the top of the regenerator a CO₂ rich gas, comprising CO₂ and steam, was obtained and from the bottom of the regenerator a CO₂ lean absorbent composition was obtained. The CO₂ lean absorbent composition was recycled via a cooling coil and a pump to the top of the absorber.

The absorber was operated at a temperature of around 40° C. and contained a stainless steel packing (EX SS316L structured laboratory packing made by Sulzer Chemtech Ltd) filling about 97% of its volume. The regenerator was operated at a temperature of around 120° C. and also contained a stainless steel packing (EX SS316L structured laboratory packing made by Sulzer Chemtech Ltd) filling about 87% of its volume. At the top of the absorber and the regenerator condensors were applied to reduce water and amine losses from the system. The unit ran continuously and comprised an automatic water adding supply to maintain the absorbent composition. The CO₂ and water content in the feed gas stream, CO₂ lean gas stream, and CO₂ rich gas stream were measured with gas chromatography (GC). The Energy required to remove CO₂ (MJ/kg CO₂) was determined by dividing the energy (MJ) added per hour via the electric heater at the bottom of the regenerator by the amount (kg) of CO₂ generated per hour at the top of the regenerator. The regenerated moles of CO₂ per kg absorbent composition were determined by calculating the amount (moles) of CO₂ generated per hour at the top of the regenerator divided by the amount (kg) of absorbent composition entering the regenerator. The delta loading was determined by dividing the amount of moles CO2 per hour retrieved via the CO2 rich gas obtained from the regenerator by the amount of moles amine per hour entering the regenerator in the CO2 rich absorbent composition, i.e. it indicates how many moles of amine were needed to regenerate 1 mole of CO2.

The percentage CO₂ recovery (%) was determined according to the following formula:

$$\frac{liter {CO}_{2 ( feed gas )} - liter {CO}_{2 [ CO 2 - lean gas ]}}{liter {CO}_{2 ( feed gas )}} * 100$$

TABLE I
Efficiency (MJ/kg CO₂)

| | | | Process conditions at 90% removal | | | | |
|---|---|---|---|---|---|---|---|
| | | | Energy | | | | |
| | | | required | Density | Regenerated | Delta | |
| | | Required | to remove | CO₂ lean | moles of | loading | |
| | Aqueous absorbent | Liquid | CO₂ | absorbent | CO₂ per kg | (mol | CO₂ |
| Exp | composition | flow | (MJ/kg | composition | absorbent | CO₂/mol | recovery |
| No. | (wt %/wt %) | (kg/hr) | CO₂) | kg/l | composition | amine) | (%) |
| Feed gas flow 645 nl/hr | | | 9.2% CO₂ | | | | |
| A | DMAPAPA/DMMEA 20/20 | 0.92 | 3.77 | 1.008 | 2.74 | 0.80 | 90.61 |
| B | DMAPAPA/DMMEA 25/25 | 0.88 | 3.47 | 1.009 | 2.82 | 0.64 | 93.29 |
| 1 | DMAPAPA/DMMEA 30/20 | 0.92 | 3.37 | 1.017 | 2.83 | 0.67 | 93.20 |
| Feed gas flow 400 nl/hr | | | 9.2% CO₂ | | | | |
| C | DMAPAPA 40% | 0.60 | 5.05 | 1.030 | 2.53 | 1.05 | 88.58 |
| D | DMAPAPA/DMMEA 20/20 | 0.59 | 4.87 | 1.016 | 2.65 | 0.48 | 89.22 |
| E | DMAPAPA/DMMEA 25/25 | 0.57 | 3.87 | 1.020 | 2.82 | 0.63 | 91.25 |
| 2 | DMAPAPA/DMMEA 30/20 | 0.57 | 3.73 | 1.025 | 2.87 | 0.69 | 93.69 |
| Feed gas flow 750 nl/hr | | | 4.6% CO₂ | | | | |
| F | DMAPAPA/DMMEA 25/25 | 0.53 | 4.60 | 1.007 | 2.79 | 0.64 | 88.21 |
| 3 | DMAPAPA/DMMEA 30/20 | 0.53 | 4.42 | 1.021 | 2.72 | 0.68 | 89.68 |

1. An absorbent composition for use in removing CO₂ and/or H₂S from a gas comprising CO₂ and/or H₂S, the absorbent composition comprises: a polyamine, a monoamine and water, wherein the polyamine comprises a polyamine having 3 to 5 amine functions and has a molecular weight of less than 200 g/mol; wherein the monoamine comprises a tertiary monoamine; and wherein the weight ratio of the polyamine having 3 to 5 amine functions to the tertiary monoamine is more than 1:1.

2. The absorbent composition of claim 1, wherein the polyamine having 3 to 5 amine functions is a non-cyclic polyamine having 3 to 5 amine functions and/or the tertiary monoamine is a non-cyclic tertiary monoamine.

3. The absorbent composition of claim 2, wherein the polyamine comprises a polyamine having 3 to 4 amine functions.

4. The absorbent composition of claim 3, wherein the total weight percentage of polyamine and monoamine is less than or equal to 70 wt % based on the total absorbent composition.

5. The absorbent composition of claim 4, wherein the polyamine having 3 to 5 amine functions is a polyamine of formula I: 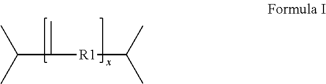
wherein each R1 independently represents a substituted or non-substituted alkylene group comprising 1 to 6 carbon atoms; wherein each R2 independently represents hydrogen-or a hydrocarbon group comprising 1 to 12 carbon atoms; and wherein x can be 1,2 or 3.

6. The absorbent composition of claim 5, wherein the polyamine comprises diethylenetriamine, dimethylaminopropylaminopropylamine or a combination thereof.

7. The absorbent composition of claim 6, wherein tertiary monoamine is a monoamine of formula II: 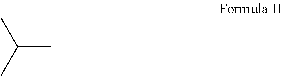
wherein each R3 independently can represent a hydrocarbon group comprising 1 to 6 carbon atoms.

8. The absorbent composition of claim 7, wherein the monoamine comprises dimethylethanolamine, diethylmonoethanol amine or a combination thereof.

9. A process for removing CO₂ and/or H₂S from a gas comprising CO₂ and/or H₂S, the process comprising the steps of:
(a) contacting the gas in an absorber with an absorbent composition wherein the absorbent composition absorbs at least part of the CO₂ and/or H₂S in the gas, to produce a CO₂ and/or H₂S lean gas and a CO₂ and/or H₂S rich absorbent composition;
(b) removing at least part of the CO₂ and/or H₂S from the CO₂ and/or H₂S rich absorbent composition in a regenerator to produce a CO₂ and/or H₂S rich gas and a CO₂ and/or H₂S lean absorbent composition;
wherein the absorbent composition is an absorbent composition as claimed in claim 1.

10. The process of claim 9, wherein the CO₂ and/or H₂S lean absorbent composition obtained in step b) is recycled to step a) to be contacted with the gas in the absorber.

11. The process of claim 10, wherein the CO₂ and/or H₂S rich gas obtained in step b) is compressed to a pressure in the range of from 20 to 300 bar.

12. The process of claim 11, wherein compressed CO₂ and/or H₂S rich gas is injected into a subterranean formation.

13. A process, comprising: using a tertiary monoamine as an accelerator for accelerating the removal of CO₂ and/or H₂S from a CO₂ and/or H₂S containing polyamine having 3 to 5 amine functions.

14. The process of claim 13, wherein the weight ratio of the polyamine having 3 to 5 amine functions to the tertiary monoamine is more than 1:1 and wherein the polyamine having 3 to 5 amine functions and the tertiary monoamine are comprised in an aqueous amine solution.
